(19) United States
(12) Patent Application Publication
(10) Pub. No.: US 2015/0328044 A1
(43) Pub. Date: Nov. 19, 2015

(54) Guide Device For A Liquid Dispenser

(71) Applicant: GLAXO GROUP LIMITED, Brentford Middlesex (GB)

(72) Inventors: James Coop, Research Triangle, NC (US); Gary Thomas CROSBY, Ware, Hertfordshire (GB); Howard Peter DUFFIELD, Ware, Hertfordshire (GB); Mark Andrew Hailey, Ware, Hertfordshire (GB); Mark Teucher, Bristol (GB); James WEST, Bristol (GB)

(21) Appl. No.: 14/649,561

(22) PCT Filed: Dec. 18, 2013
(86) PCT No.: PCT/EP2013/077131

§ 371 Date: Jun. 4, 2015

Related U.S. Application Data
(60) Provisional application No. 61/739,128, filed on Dec. 19, 2012.

(51) Int. Cl. A61F 9/00

(52) U.S. Cl. CPC A61F 9/0026

(57) ABSTRACT

A guide device for assisting the application of a liquid from a dispenser to the eye of a subject has a mount to hold the liquid dispenser in the guide device, a rest to align the guide device with the subject head, and an indicator, selectively visible to indicate when the guide device is tilted at or beyond a minimum guide angle. Preferably, the indicator is selectively visible to indicate when the guide device is tilted at, or between, the minimum guide angle and a maximum guide angle.

Said minimum guide angle corresponds with a minimum predetermined tilt of the subject head suitable for delivery of the liquid from the dispenser. The maximum guide angle corresponds with a maximum predetermined tilt of the subject head suitable for delivery of the liquid.

The present invention relates to a guide device for aiding a subject in the application of a liquid from a dispenser to the subject's eye or for aiding a third party in the application of the liquid to the subject's eye. In particular, the invention ensures reliable delivery of a droplet of medication by a patient, or a third party, from an eye dropper to the eye of the patient.

Reliable delivery of topical medication to a patient's eye is problematic; good visual acuity and good manual dexterity are required for correct administration by existing droppers such as single-use blow fill sealed nebules, or multi-dose dropper bottles.

A number of factors contribute to this requirement; because droplets fall from the dispenser onto the surface of the eye, the dropper must be steadily aligned with the eye. At the same time, the outlet must be spaced apart from the eye to avoid contact damage to the eye, or contamination of the dropper outlet. The patient head must also be tilted backwards, i.e. angled in the sagittal plane away from the vertical, to maximise the horizontal aspect of the eye surface available for the drop to fall on. A further complication is that the eyelid needs to be held open as widely as possible to again maximise the area available for the drug to fall on.

Somewhat paradoxically, however, patients who require eye medication often suffer reduced visual acuity due to the disease being treated. Furthermore, because such patients are often drawn from older population groups, they are more likely to suffer reduced dexterity due to aging related conditions such as osteoarthritis.

As a result, patients often fail to correctly self-administer the drug to their eyes. This is especially problematic because eye medication is delivered in small doses, typically 40 microlitres. These small doses leave little indication if they are delivered outside of the eye, a problem compounded by tearing of the eye during drug administration.

This has the result that it is practically impossible to reliably identify when medication has not been delivered to the eye. Absent this feedback, it is all too possible for a patient to repeatedly fail to deliver medication correctly to their eye(s).

It can also be added that, even where the drug is administered by a third-party such as a care-giver, there is still a serious risk that incorrect delivery of the drug is not identified for the reasons set out above.

Hence there is a requirement for a guide device which enables patients or care-givers to reliably deliver a drug to the eye which does not require good visual acuity or good dexterity.

Furthermore, it is preferable to provide such a guide device without the use of electronic or electrical components. Electronic and electrical components are expensive and difficult to recycle at the end of a product's life.

### SUMMARY OF THE INVENTION

According to a first aspect of the present invention, there is provided a guide device for assisting the application of a liquid from a dispenser to an eye of a subject comprising;

a mount, to hold the dispenser in the guide device,
a rest, to align the guide device with the subject head such that the mount locates an outlet of the dispenser in fixed relationship with the subject head,
an indicator, which is selectively visible to indicate when the guide device is tilted at or beyond a minimum guide angle,
wherein said minimum guide angle corresponds, when the guide device is aligned with the subject head via the rest, with a minimum predetermined tilt of the subject head suitable for delivery of the liquid from the dispenser outlet to the subject eye.

Preferably, the device is entirely mechanical in operation, requiring no electrical or electronic components.

Preferably, the rest is an eyecup, adapted to rest about an eye of the subject. Preferably, the eyecup surrounds and shrouds the eye when the guide device is aligned with the subject head.

Preferably, the indicator is only visible when the guide device is tilted at or beyond the minimum guide angle.

Suitably, the indicator is selectively visible to indicate when the guide device is tilted at or between the minimum guide angle and a maximum guide angle, wherein the maximum guide angle corresponds, when the guide device is aligned with the subject head via the rest, with a maximum predetermined tilt of the subject head suitable for delivery of the liquid from the dispenser outlet to the subject eye.

Preferably, the minimum guide angle corresponds, via alignment of the guide device with the subject head via the rest, with a minimum tilt of the subject head of about 45° to the horizontal. More preferably 45°.

Preferably, the minimum guide angle corresponds, via alignment of the guide device with the subject head via the rest, with a minimum tilt of the subject head of about 50°, more preferably 50°.

Preferably, the minimum guide angle and maximum guide angle are separated to give a range of about 10° between the minimum predetermined tilt of the subject head and the maximum predetermined tilt of the subject head, more preferably 10°.

Preferably, the indicator is illuminated.

Preferably, the indicator is illuminated by channelling ambient light to the target. Preferably by use of a light-guide.

Suitably, the selectively visible indicator is only visible when the guide device is tilted at an angle at or between the minimum guide angle and the maximum guide angle.

Suitably, the guide device further comprises a moveable shroud which renders the indicator selectively visible.

Suitably, the shroud is mounted for movement within the guide device driven by the weight of the shroud.

Suitably, the shroud comprises at least one pendulum.

Suitably, the at least one pendulum is an inverted pendulum, such that when the guide device is located at or beyond the minimum guide angle, the centre of gravity of the pendulum lies above the pendulum pivot.

Suitably, the shroud further comprises a shutter.

Preferably, the shroud comprises a first pendulum and a second pendulum. Preferably, each pendulum comprises a shutter.

Suitably, the indicator is selectively visible to the eye of the subject and wherein the shroud is selectively interposed between the subject eye and the indicator.

Preferably, when the shroud is interposed between the subject eye and the indicator, the indicator is invisible.

Suitably, the indicator is selectively visible to the eye of the subject.

Suitably, the indicator provides a target which directs the subject's gaze upwards.

Preferably, the rest comprises an eyecup which surrounds the subject eye, and the target is located within a cavity defined by the cup.

Preferably, the cup is treated to reduce transmission of ambient light, thereby enhancing contrast between the cup and the target.

Preferably, the cup is provided with a lens between the target and the subject eye to magnify the target.

Suitably, the indicator provides a crescent shaped target, located within the device to lie above and concentric with the outlet of a dispenser mounted within the guide device.

Preferably, the target is coloured green for patients suffering with damaged central vision, to improve visibility.

Suitably, the indicator is selectively visible to a third party.

Suitably, the guide device further comprises an actuator for operating the liquid dispenser.

Suitably, the actuator is adapted to convert an arcuate motion applied by an actuator lever into a substantially linear motion applied to the dispenser.

Preferably, the actuator comprises a Watt's linkage.

According to a second aspect of the present invention, there is provided an eye dropper device comprising the guide device of claim 1 and a liquid dispenser.

Suitably, the dispenser is a metered droplet dispenser for dispensing metered droplets of a liquid medication.

Other aspects and exemplary features of the invention are to be found in the exemplary embodiments which will now be described, by way of example only, with reference to the accompanying Figures of drawings.

### BRIEF DESCRIPTION OF FIGURES OF DRAWINGS

FIG. 1 shows a first perspective view on an eye dropper device according to the present invention, further comprising a guide device according to the present invention.

FIG. 2 shows a second perspective view on the eye dropper.

FIG. 3 shows a sectioned view on the eye dropper in a predetermined dispensing orientation.

FIG. 4 shows an exploded view of the eye dropper device.

FIG. 5A shows an indicator assembly of the eye dropper device with certain elements omitted for clarity.

FIG. 5B shows, schematically, a cross-section through the indicator assembly, of the eye dropper device.

FIG. 6 shows a view on the eyecup cavity of the dropper device.

FIG. 7a shows a sectioned view of the dropper device, absent part of the housing, in a first, sub-optimal, delivery orientation.

FIG. 7b show the indicator assembly of the device in the first, sub-optimal, delivery orientation.

FIG. 8a shows a sectioned view of the dropper device in an optimal, delivery orientation.

FIG. 8b show the indicator assembly of the device of FIG. 8a in the optimal, predetermined, delivery orientation.

FIG. 9a shows a sectioned view of dropper device in a second sub-optimal orientation.

FIG. 9b show the indicator assembly of the device of FIG. 9a in the second sub-optimal orientation.

FIG. 10A shows a perspective view on a first prototype guide device.

FIG. 10B shows a second perspective view on the device of FIG. 10A.

FIG. 10C shows a perspective view on the upper part of the guide device of FIGS. 10A and 10B, sectioned to show the internal construction of part of the device.

FIG. 11A shows a perspective view on a second prototype guide device.

FIG. 11B shows a second perspective view on an upper part of the guide device of FIG. 11A, sectioned to shown the internal construction of part of the device.

### NOTE ON FIGURES

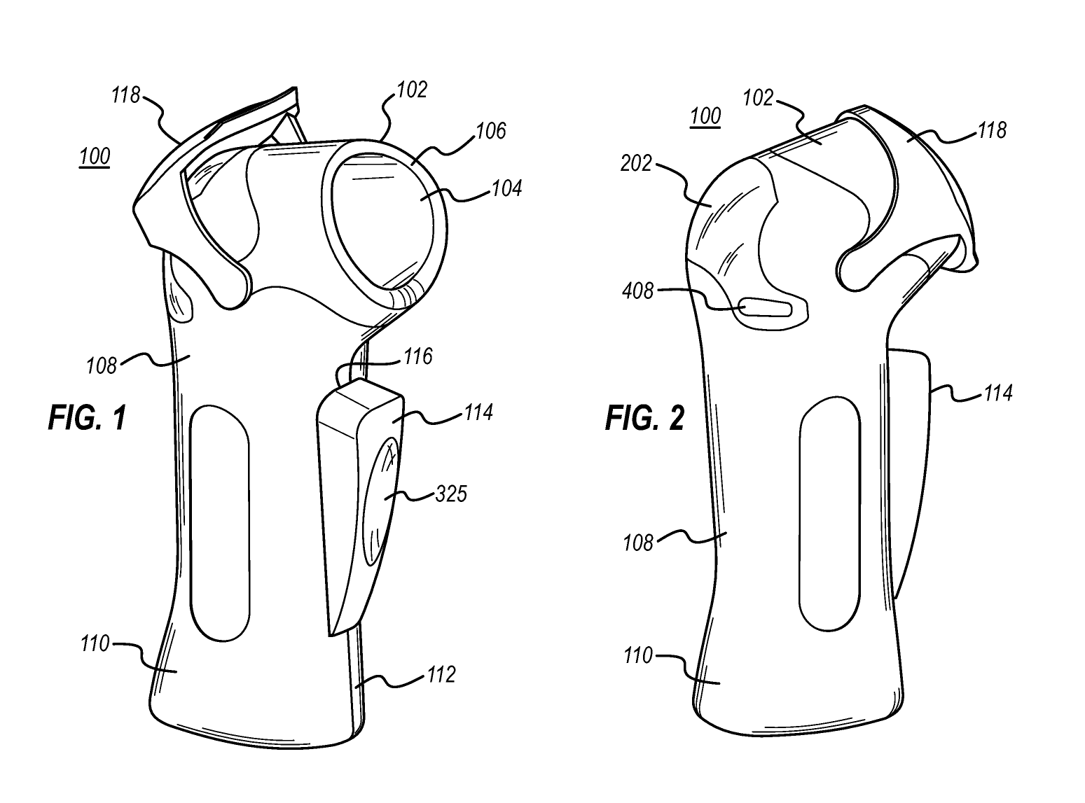

FIGS. 1 through 9b are based upon engineering drawings used for development of the device. Hence the drawings are to scale and representative of the geometry of a guide device according to the present invention, and of an eye dropper device according to the present invention.

### DETAILED DESCRIPTION OF THE EXEMPLARY EMBODIMENTS OF THE INVENTION

Referring now to FIG. 1, there is shown a first perspective view on an eye dropper device 100. The device 100 comprises an eyecup 102 which defines an open cavity 104. The cup 102 provides a continuous rim 106 which is sized and shaped to surround a patient's eye when in abutment with the patient's face, presenting the cavity to the eye. The eyecup 102 thereby provides a rest 102, shaped to orient the device 100 in a single position relative to a patient's head to provide a reliable datum. In use, the rest 102 spaces a dispenser outlet (not shown), located within the cavity 104, in fixed relationship with the patient's head, and the orbit of the patient's eye. In more detail, the dispenser outlet is held apart from the eye to prevent the outlet from contacting the eye which prevents contact damage to the surface of the eye and also avoids contamination of the nozzle.

A continuous annular cup is preferred as it provides a barrier around the eye which isolates the eye and dispenser outlet from the external environment. This aids delivery of a droplet from the outlet to the patient eye by providing a region of still air within the cavity as well as inhibiting the entry of contaminants, such as dust, into the cavity.

The eyecup 102 is attached to a housing 108, comprising a first half 110 and second half 112 which are joined together. An actuation lever 114 projects from the housing via a lever aperture 116.

A hinged lid 118 is permanently, pivotally, mounted to the housing 108 so that it can be pivoted from a closed position, shown in FIG. 2 to an open position, as shown in FIG. 1, but cannot be removed from the device 100. In the open position shown in FIG. 1, the cavity 104 is fully accessible.

Turning now to FIG. 2, which shows a second perspective view on the eye dropper device 100, the lid 118 is pivoted over the rim of the eyecup to close the cavity 104. This prevents ingress of contaminants into the cavity 104 when the device 100 is not in use which might otherwise transfer into the patient eye upon use, causing tearing, and inhibiting efficient delivery of a drug to the eye.

The housing 108 further comprises a light-guide panel 202 which is a light gathering component. This is made from a transparent base polymer, preferably Poly(methyl methacrylate) also known as PPMA or Acrylic. The light-guide panel 202 is pigmented with a fluorescent colour, preferably green, for reasons that will be discussed hereinafter.

The light-guide panel 202 can alternatively be manufactured from polycarbonate, or polished polypropylene.

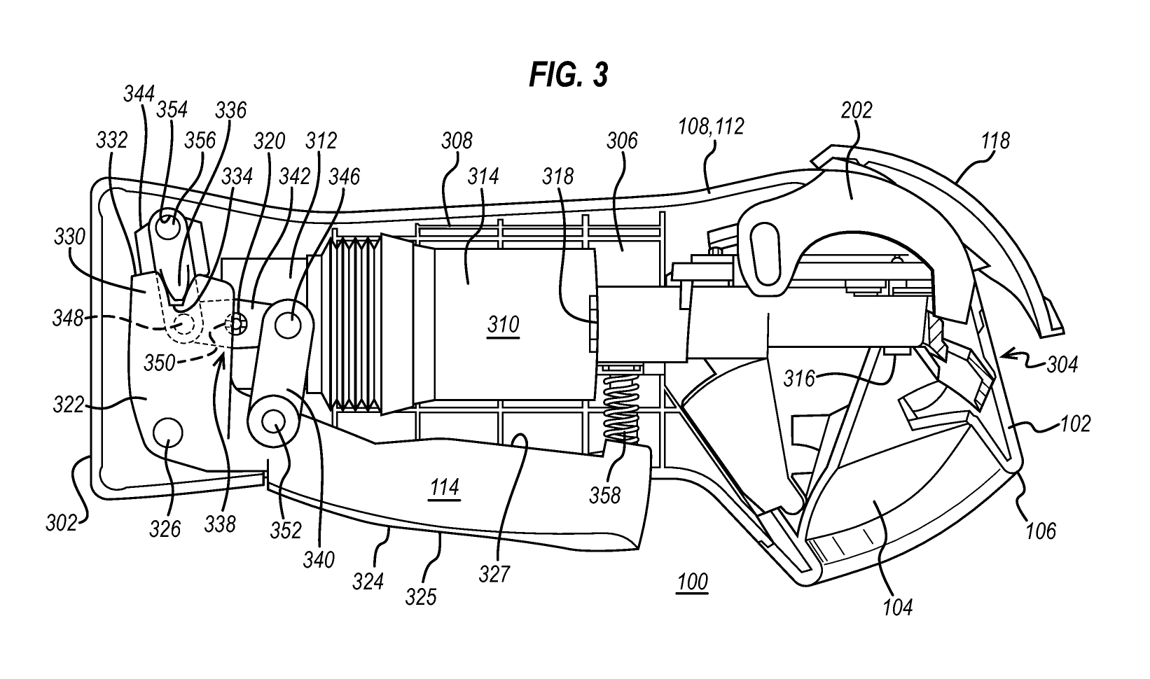

Turning now to FIG. 3, there is shown a view on the eye dropper device 100 in a predetermined dispensing orientation, optimised for delivery a droplet of drug to the patient eye. The first housing half 110 is omitted to allow the construction of the device 100 to be better understood.

The device housing 108 extends from a base 302 to a distal, dispensing, end 304 and defines an enclosed elongate cavity 306. The housing is locally reinforced with internal ribs 308, and houses and supports a metered liquid droplet dispenser (MLDD) device 310, which is set forth in more detail in patent application PCT/EP2013/068316.

In brief, the metered liquid droplet dispenser comprises a reservoir/drive piston 312 which is slidably received within a housing 314 of the MLDD. A predetermined withdrawal of the drive piston 312 from the housing 314 energizes, and primes, the MLDD. Once the piston 312 is withdrawn the predetermined distance, the MLDD operates autonomously to dispense a metered droplet of liquid medication independently of any further movement of the piston/drive piston. The droplet is dispensed from a nozzle 316, located at a distal end of the MLDD relative to the reservoir/drive piston assembly 312, which is sealed by a sprung tip seal (not shown). During the autonomous delivery phase of operation, the tip seal is opened by a high pressure hydraulic circuit that uses liquid medication as its working fluid. At the same time, a dose of the liquid medication is dispensed via a low pressure circuit, which operates independently of the high pressure circuit at dosing. Dosing is terminated by venting the high pressure hydraulic circuit while the low pressure circuit is still pressurised, ensuring that there is always a positive pressure at the nozzle 316 when open.

The drive piston 312 is returned to its rest position by external force i.e. it is pushed back into the MLDD housing 314.

The cavity 306 defined by the eye dropper housing 108 is shaped to conform to the external shape of the MLDD 310 to support it within the housing 108, and to minimise the overall envelope of the device 100. The MLDD housing 314 is also provided with a pair of housing actuation lugs 318, of which only one is visible in FIG. 3. The lugs 318 are provided on either side of the dispenser device housing 314 so that each lug is received in a cooperating slot in a respective half 110, 112 of the eye dropper housing 108. The actuation lugs 318 engage the slots to lock the MLDD 310 relative to the eye dropper housing 108.

The reservoir/drive piston 312 of the MLDD 310 provided with a pair of reservoir actuation lugs 320, of which only one is visible in FIG. 3. The lugs 320 are provided on either side of reservoir drive piston 312, diametrically opposed to each other. The lugs 320 are cruciform in cross-section, and provide a pivot for a Watt's linkage actuation mechanism, which converts pivoting motion applied by the actuation lever into a substantially linear motion which is applied, via the lugs 320, to the reservoir/drive piston 312.

The actuation lever 114 is L shaped, comprising a minor base limb 322 and a main elongate limb 324. A pair of cylindrical pivot recesses 326 is provided on a common axis, one located on each side of the lever 114 at the junction of the minor and major limbs 322,324. Each recess 326 receives a lever spindle from a respective housing half (not shown in FIG. 3) so that the lever 114 is mounted within the housing 108, pivotable about the lever spindles.

The actuation lever 114 is located on the lower side of the device 100 in the approximately horizontal orientation used for dispensing medication from the MLDD 310. The lever 114 is pivoted near the housing base 302 and extends towards the distal dispensing end 304 so that the device 100 can be gripped by a patient using a so called crush grip wherein the patient curls the fingers of the hand over the upper surface of the device (i.e. the opposite side to the lever) and supports the device 100 on the heel pad of their hand. The patient can then place their thumb along the actuation lever 114 and actuate the device 100 by bringing the thumb towards the fingers, squeezing the device to drive the lever 114 inwards. This efficiently uses the strength available in the patient's hand, ensuring that they do not need to overexert themselves during actuation of the device 100. This avoids shaking and a consequent risk of non-dosing.

The lever 114 is provided with an elongate indentation 325 to locate the patient thumb a safe distance from the patient eye. This prevents conflict between the patient thumb and face at dosing which would otherwise move the device 100 relative to the patient head, disrupting the dosing process.

The internal surface 327 of the lever 114 is shaped so that when it is fully depressed, as shown in FIG. 3, it conforms to the outer mould line of the MLDD 310, thereby minimising the volume of the device 100.

The minor limb 322 of the actuation lever 114 extends at about 90° to the major limb 324 so that it lies generally parallel to the base 302 of the device housing 108. The minor limb 322 is forked so that a first prong and second prong 330 lie on either side of the drive piston 312 of the MLDD 310 in the assembled state shown.

The distal end 332 of each prong 330 is notched to define an involute tooth space 334. This tooth space 334 is shaped to receive an involute tooth 336 which projects from a Watt's linkage 338, also known as a parallel linkage, 338 which is pivotally mounted to the housing 108, within the cavity 306.

The Watt's linkage 338 comprises, on both side of the MLDD 310, three rods, 340, 342, 344, attached in sequence. The central rod 342 is attached to a first end rod 340 by a first hinge 346 and to a second end rod 344 by a second hinge 348. The second hinge 348 is shown in dashed outlined in FIG. 3 as it is hidden by the prong 330 of the lever 114.

The end rods 340, 344 of the linkage 338 are of substantially equal length.

The central rod 342 is provided with a central circular hole 350 which receives the reservoir actuation lug 320 and allows the linkage 338 to pivot thereabout.

The first rod 340 is provided at a first end with a cylindrical aperture 352, which engages a spindle (not shown) mounted to the housing 108. The third rod 344 is pivotally mounted, via an integral cylindrical aperture 354 to the housing 108 via a spindle 356.

To actuate the dropper device 100 to dispense a droplet of medication, a user squeezes the lever 114 into the device housing 108, which causes the lever 114 to rotate about the lever pivot 326. This motion causes the involute tooth space 334, located at the end of the lever minor limb 322, to arc about the same pivot 334 towards the base 302 of the housing 108. The tooth space 334 drives the involute tooth 336 of the Watt's linkage 338 to pivot the third rod 344 about its spindle 356, towards the base 302 of the device 100. The interaction of the three rods 340, 342, 344 of the linkage 338 applies a substantially linear force to the drive piston 312 of the MLDD 310 via the reservoir actuation lugs 320, which withdraws the reservoir/drive piston 312 from the MLDD housing 314. As set forth previously, this primes and energises the MLDD 310 for subsequent autonomous operation.

Upon release of the lever 114, a return spring 358, located between the MLDD 310 and the lever 114 returns the lever 114 to an ‘at-rest’ position. The returning lever 114 drives the Watt's linkage 338 in the opposite direction to actuation, withdrawing the MLDD drive piston 312 to restore the MLDD 310 to its rest state. Use of an external actuation and return mechanism minimises the complexity of the MLDD 310. This allows the ergonomics of device actuation e.g. actuation force, to be modified relatively simply, without having to redesign the MLDD 310.

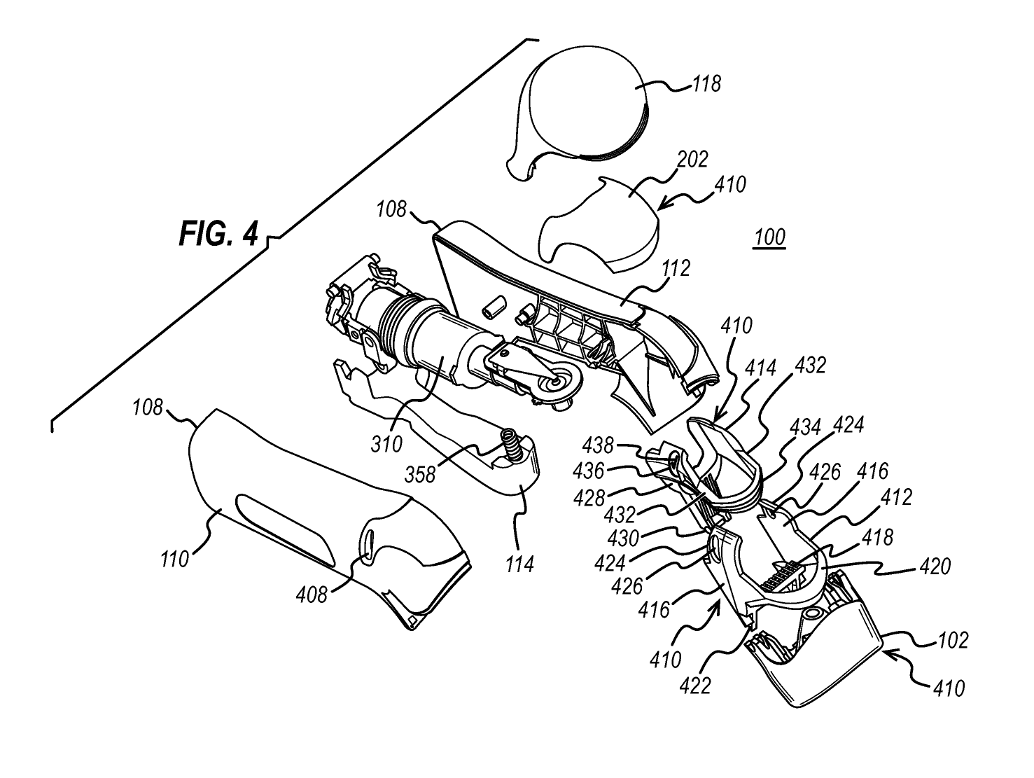

Turning now to FIG. 4, there is shown an exploded view of the eye dropper device 100. A window aperture 408 is provided in each housing half 110, 112 (only one visible in FIG. 4) for reasons that will be explained below.

The device 100 comprises an indicator assembly 410 which comprises the eyecup 102, the light-guide panel 202, and a first pendulum 412 and second pendulum 414.

The first pendulum 412 comprises a pair of counter weights 416 which are held, spaced from one another, by a pivot bar 418 and an arcuate first shutter 420. The bottom of each weight 416 is bar is provided with a conical pivot 422, of which only one is visible in FIG. 4. Hence the weight 416 and pivot 422 define an inverted pendulum 412.

Each weight 416 is provided, at a distal end from the pivot 422 with a vertical projection 424 which defines an elongate aperture 426.

The second pendulum 414 comprises a single weight 428. First and second conical pivot 430 are provided at the base of the weight 428, so that the weight 428 and pivots 430 define an inverted pendulum 414. A pair of spaced apart arms 432 project forwards from the top of the weight and support an arcuate second shutter 434 which extends between the arms 432.

Each arm 432 is provided with an indentation 436, which is marked with a high contrast tick symbol 438.

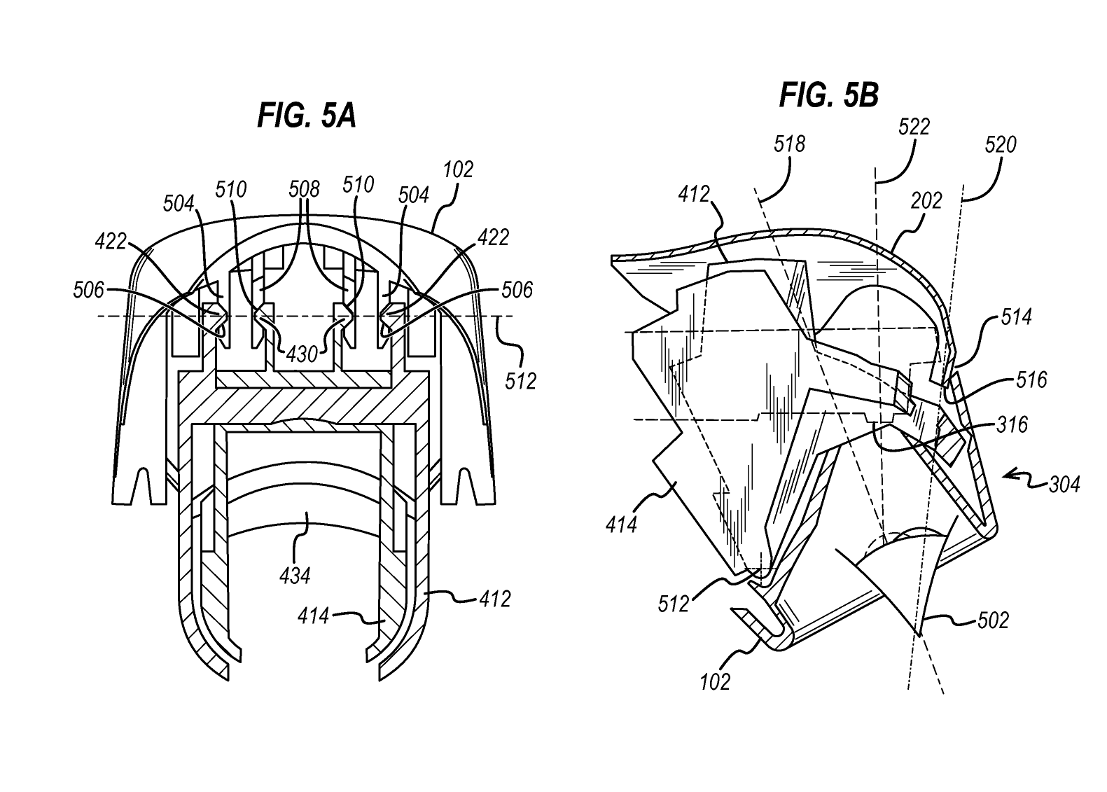

Turning now to FIGS. 5A and 5B, FIG. 5A shows a view on the underside of the first pendulum 412, second pendulum 414, and eyecup 102 in an assembled state. The first and second pendulums 412, 414 are shaded to clarify the construction of the assembly.

FIG. 5B shows, schematically, a cross-section through the assembly of FIG. 5A, and further includes other elements of the eye dropper device 100, particularly the light-guide panel 202, and the nozzle 316 end of the MLDD 310. FIG. 5B also shows a patient eye 502, aligned with the dispensing device 100.

As assembled, the first pendulum 412 is mounted to the eyecup 102 for pivoting motion about a first pair of sprung arms 504. Each arm has a conical recess 506 which receives the conical pivot 422 of the first pendulum. In the unassembled state, the distance between these conical recesses 506 is greater than the distance between the conical pivots 422 of the first pendulum 412. This ensures that, when assembled, the arms 504 are sprung inwards, i.e. towards each other, so that there is no end float between the first pendulum 412 and the eyecup 102.

As assembled, the second pendulum 414 is mounted to the eyecup 102 for pivoting motion about a second pair of sprung arms 508 which lie inboard of the first pair of sprung arms 504. Each arm 508 provides a conical recess 510 which receives the conical pivot 430 of the second pendulum 414. In the unassembled state, the distance between the conical recesses 510 of the inner sprung arms 508 is less than the distance between the conical pivots 430 of the second pendulum 414. This ensures that, when assembled, the arms 508 are sprung outwards, i.e. away from each other, so that there is no end float between the second pendulum 414 and the eyecup 102.

The lack of end float in the assembly of eyecup 102 and the pendulums 412, 414 ensures that the shutters 420,434 of each pendulum 412, 414 are correctly aligned and that the pendulums 412, 414 operate consistently under a constant friction at the interface between the conical pivot and recess. Conical pivots are used to minimise any stiction between the pendulums 412, 414 and the eyecup 102 in order to ensure consistent and reliable operation of the pendulums 412, 414, as set forth below.

The conical pivots of each pendulum 412, 414 are arrayed to provide pivoting of each pendulum about a common axis 512 defined by the pivot of both pendulums.

Turning to FIG. 5B, the light-guide panel 202 is shaped to conform to the outer form of the housing 108 at the dispensing end 304 of the dropper device 100, and is positioned on the rear of the device housing 108. The panel 202 has a large surface area in order to collect as much ambient light as possible but narrows towards the uppermost end of the device i.e. at the dispensing end 304. The panel 202 enters the housing 108 via an aperture 514 formed in the housing 108. The light-guide panel 202 is shaped to define a thin, crescent-shaped planar face 516 which lies within the housing centred above, and about, the MLDD nozzle 316. Ambient light, collected by the light-guide panel 202 is trapped by internal reflection and directed towards the planar surface of the crescent 516. When it reaches the surface 516, it is scattered, illuminating the surface of the crescent which causes it to glow strongly.

With reference to FIG. 5B the crescent shaped planar face 516 provides an illuminated target 516 when ambient light falls on the light-guide. When visible to the patient in the dispensing attitude shown, the target 516 directs the gaze of the patient eye upwards, from the normal straight ahead gaze shown by dashed line 518, to an upward gaze shown by dashed line 520. Direction of the patient's eye upwards within its orbit has a number of benefits. The droplet is less likely to strike the cornea which is rotated upwards from the straight ahead position, out of the path of a droplet dispensed indicated by dashed line 522. The droplet instead falls on the less sensitive sclera, reducing the likelihood of blinking or tearing. A further benefit is that the upward gaze raises the upper eyelid which increases the area exposed to the droplet, and also moves the eyelashes of the upper eyelid out of the delivery path.

It will be understood that the straight ahead gaze indicated by line 518 lies at about 90° to the vertical axis of the patient's head. In the dispensing attitude shown in FIG. 5B, the patient's head is tilted backwards at about 55° to the vertical. For the MLDD 310 of the specific embodiment, the optimum delivery angle for delivery of a droplet of medication to the eye requires the patient head to be inclined in the sagittal plane at an angle of around 55° to the vertical. Acceptable results are provided with the head tilted within a range of about 10° from this value, i.e. from a minimum backward tilt of about 50° to a maximum tilt of about 60°.

Patient handling studies show that a minimum value of 45°, with a maximum value of 55°, is more ergonomically preferred, and it will be understood that the guide device of the present invention can be used with alternative liquid droplet dispenser devices to provide a eye dropper device which work at this lower range of angles of tilt of the patient head.

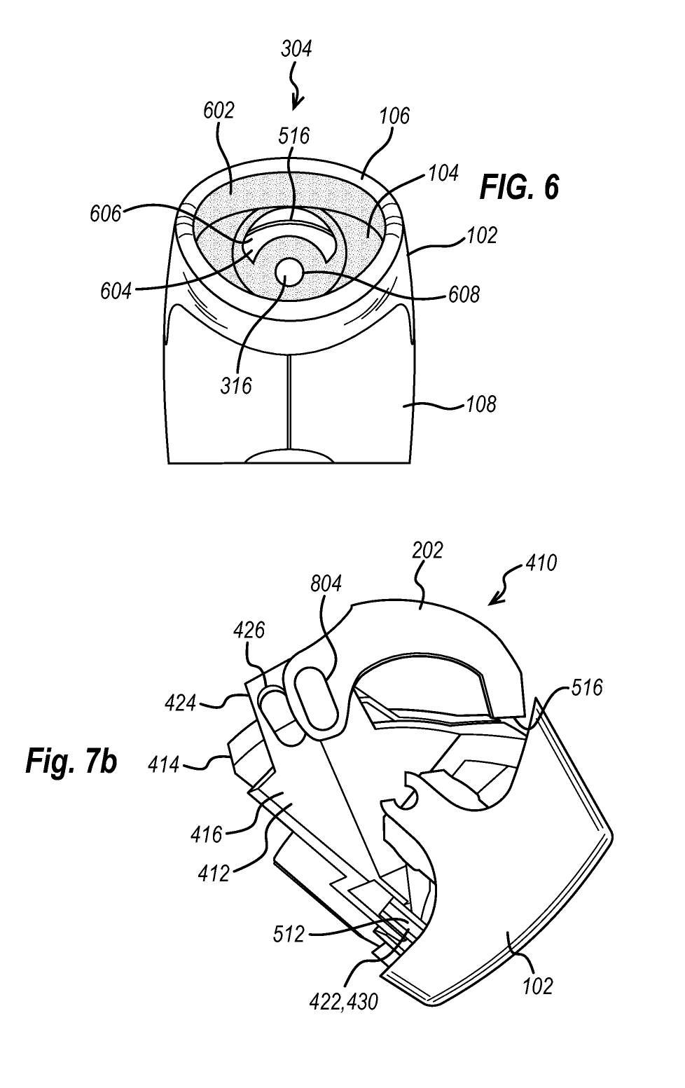

Turning now to FIG. 6, which shows a view on the cavity 104 of the device 100 of FIGS. 1, 2 and 3, it can be seen that the thin crescent shaped planar face 516 forms a highly visible, illuminated target 516 within the cavity 104.

The eyecup 102 is manufactured of transparent polypropylene to allow the user to view the target 516 through the wall of the eyecup 102. The internal surface 602 of the eyecup 102 is provided with a texturised finish which provides a matt, opaque surface, except for a locally smooth, polished, region in a window 604 adjacent the target 516. The contrast between the window 604 and the surface of rest of the cavity 104 helps to enhance visibility of the target 516 to the patient. The window 604 provides a magnifying lens via a locally convex surface 606, to increase the apparent size of the target 516, and hence the effective illuminated area, to the patient.

A Fresnel lens can be used instead of the locally convex surface to minimise the bulk of the window 604.

It is also possible to provide a window region of constant wall section, so that the target 516 is viewed at normal size.

The target 516 and target window 604 are located above an outlet aperture 608, formed in the internal surface 602 of the eyecup 102, which allows medication to fall from the nozzle 316 of the MLDD onto the patient eye. The crescent shape of the target 516 is located above the nozzle 316 as the eyecup 102 is presented to the patient eye, as shown in FIG. 6. The crescent shaped target 516 is also wrapped partially around the upper hemisphere of the nozzle outlet 316.

As discussed previously, the location of the target 516 directs the patients gaze vertically above the nozzle 316. Furthermore, the offset wrap-around location of the target 516, relative to the outlet nozzle 316 and outlet aperture 608, ensures that at least a part of the target 516 will be visible to the upper peripheral hemisphere of vision of the patient. Patients suffering from compromised central vision, as can arise from diseases such as wet age-related macular degeneration (wet AMD), are thus able to see the target with their peripheral vision, enabling direction of gaze by the target, and effective indication to the patient that the device 100 and patient head, is correctly oriented, as set forth in more detail below.

The target 516 is a crescent shape to create the largest light for the given space constraints within the device 100. The light-guide panel 202 is preferably pigmented with a fluorescent green colour to further intensify the light emitted from the target 516. Green has been found preferable for patients suffering from wet AMD who have difficulty with colours in the red area of the light spectrum.

### Use of Device

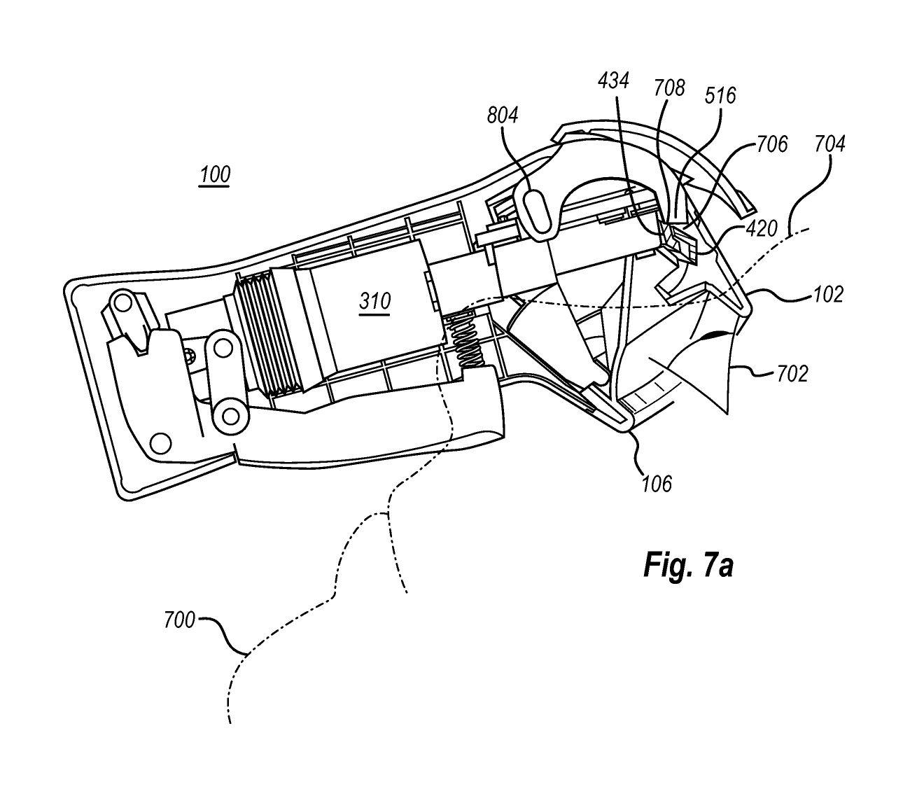

FIGS. 7a to 9b show the device 100 and the device indicator assembly 410 in a sequence of positions. In particular, FIGS. 7a, 8a and 9a show the device 100 aligned with a patient's head 704 via the cup 102 and FIGS. 7b, 8b and 9b show the indicator assembly 410 of the device 100 in isolation;

FIGS. 7a and 7b show the device 100 and the indicator assembly 410 in a first non-optimal delivery orientation.

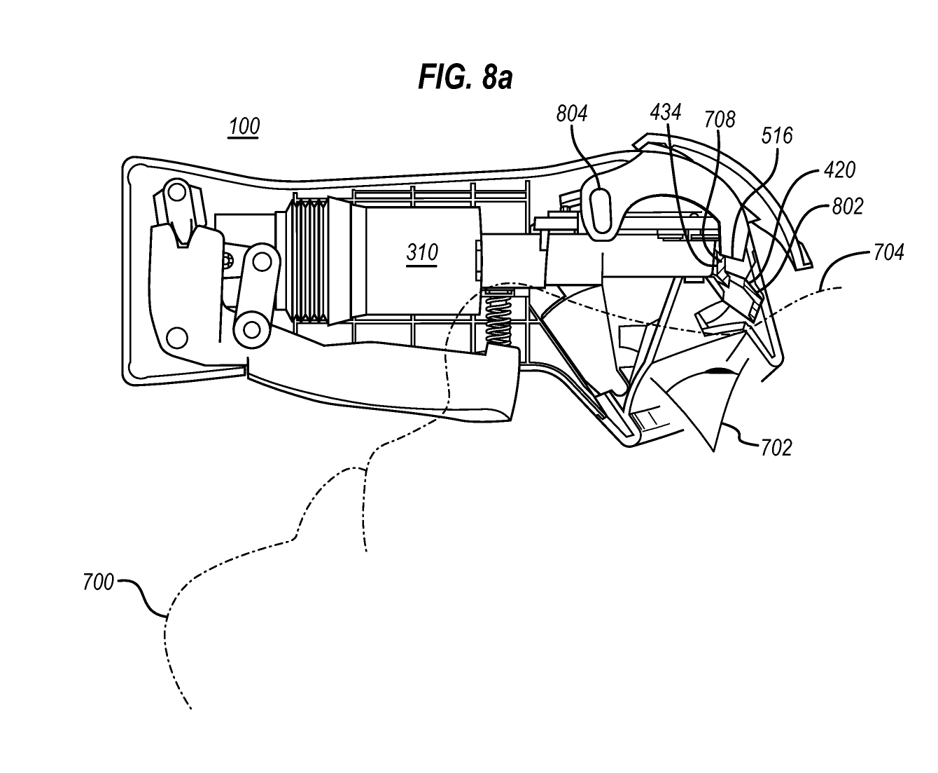

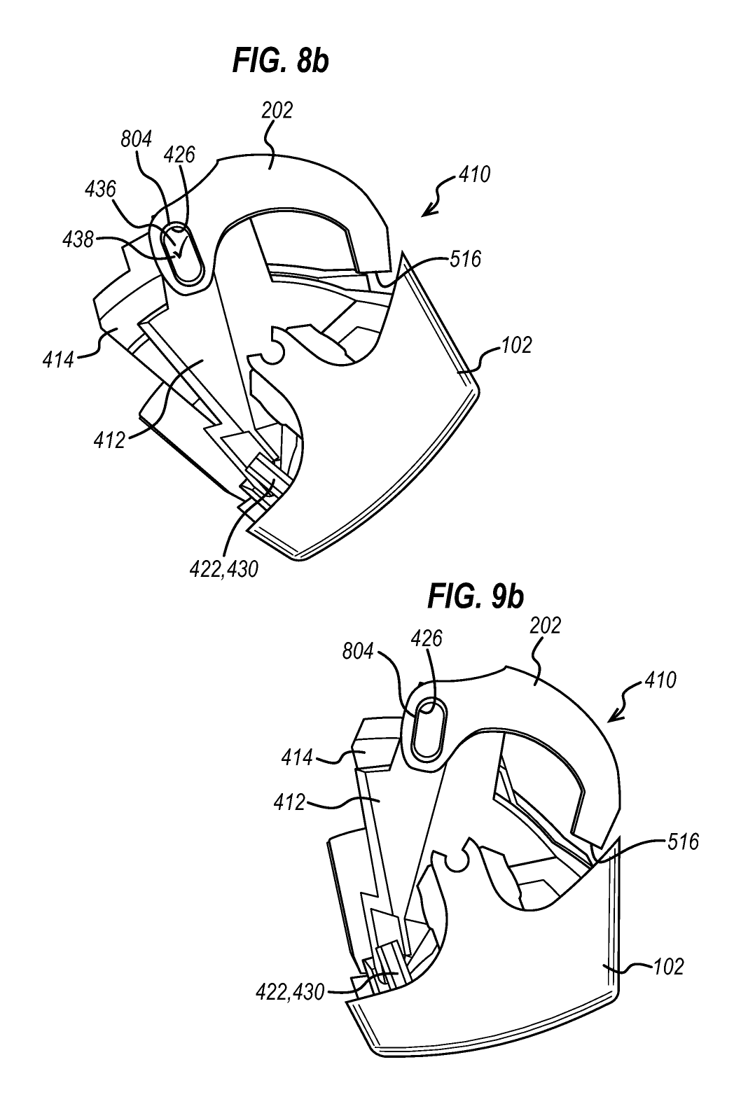

FIGS. 8a and 8b show the device 100 and the indicator assembly 410 in the predetermined delivery orientation.

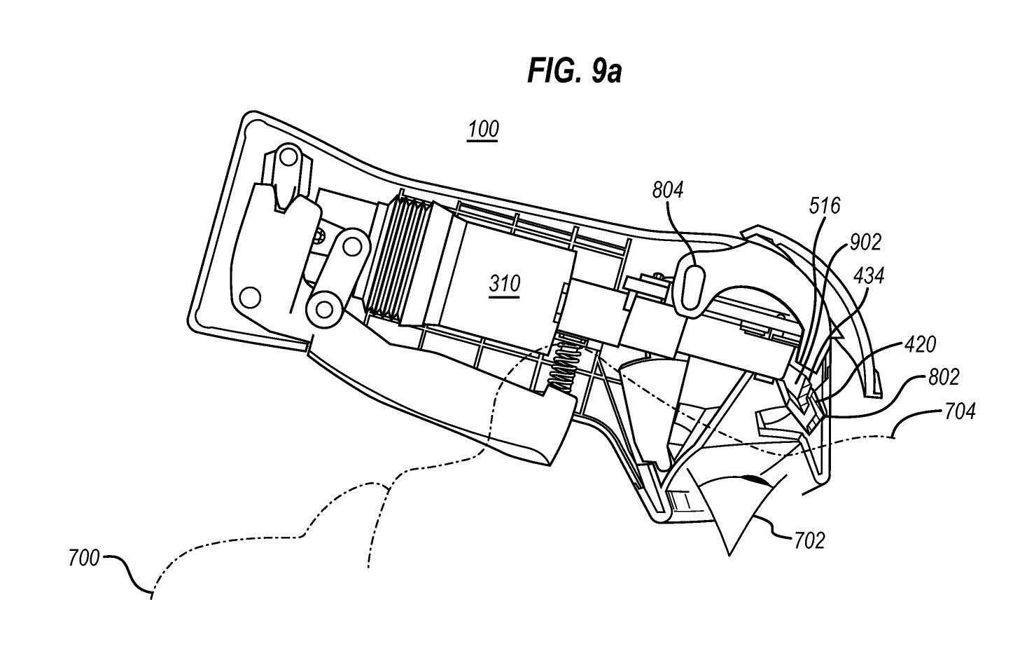

FIGS. 9a and 9b show the device 100 and the indicator assembly 410 in a second non-optimal delivery orientation.

Referring now to FIG. 7a, the user/patient 700, shown in dashed outline, has placed the eyecup 102 around his/her eye 702, to which a medication is to be applied. The eyecup rim 106 acts as a rest which aligns the dropper device 100 relative to the patient head 704 in a single predetermined position thereto, and is shaped to maintain the device 100 in this position relative to the head throughout use of the device 100.

In the present device 100 using the MLDD 310 exemplified, drug is optimally delivered to the eye when the patient head is tilted backwards at an angle between 50° and 60° to the vertical, preferably 55°.

FIGS. 7a and 7b show the device 100, and indicator assembly 410, in a first orientation of the device wherein the device is aligned with the patient head via the eyecup 102, and the device is tilted at an angle which is less than a minimum guide angle corresponding with a backward tilt of the patient head of less than 50°. In other words, in FIGS. 7a and 7b, the patient head is tilted backwards at an angle of less than the predetermined minimum delivery angle of 50°, and the device 100 is therefore tilted at an angle less than a corresponding minimum guide angle.

In this first orientation, the weight of the first pendulum 412, located above the pendulum pivot 422, ensures that the first shutter 420 is held in a rest position 706 wherein it lies interposed between the target 516 and the eye 702, rendering the target invisible to the patient. In particular, the centre of gravity of the first pendulum 412 is located such that it lies behind the conical pivots 422 i.e. towards the device base 302, for all angles of the device 100 from the rest state shown in FIGS. 1 and 2 in which the device 100 rests on its base 302, through the orientation shown in FIGS. 7a and 7b up to the minimum guide angle which corresponds, in use, to the head being tilted at an angle to the vertical of 50°, i.e. the lower, “threshold” value of the optimum delivery angle range. This arrangement ensures that gravity holds the first shutter 420 in the rest position until the device 100 is tilted to at least the minimum guide angle.

The second shutter 434 is held in a rest position 708 in a similar manner; the centre of gravity of the second pendulum 414, located above the pendulum pivot 430, is located such that it lies behind the conical pivot, i.e. towards the device base 302, to hold the shutter 434 in the rest position under the force of gravity. However the centre of gravity is located further aft relative to the pivot 430 than with the first pendulum 412. This has the effect that the centre of gravity lies behind the conical pivot 430 of the second pendulum 414 from the rest state shown in FIGS. 1 and 2 in which the device rests on its base, up to a maximum guide angle which corresponds, when the device is aligned with the patient's head, to a backward tilt of the patient head of 60° to the vertical, i.e. the maximum predetermined backward tilt of the subject head for optimal delivery. This arrangement ensures that gravity holds the second shutter 434 in the rest position until the device 100 reaches the maximum guide angle.

It will also be seen that the aperture 426 formed in the vertical projection 424 of the first pendulum 412 overlaps the arm 432 of the second pendulum 414 so that the indentation 436 formed in the second pendulum, and the tick symbol 438, is not visible via the window aperture 408 formed in the housing 108. This enables a third party, such as a care giver, to determine that the device 100 is not correctly oriented for delivery of a drug in the event that the patient is unable to use the device independently.

FIGS. 8a and 8b show the device 100 and indicator assembly 410 in a second orientation, when the patient head is oriented at an angle of between 50° and 60° to the vertical. The device 100 remains in place with the eyecup 102 located about the eye 702 to align the dropper device 100 relative to the patient head 704.

In use, as the device 100 is moved from the position shown in FIG. 7a, to the position shown in FIG. 8a, it reaches the minimum guide angle, corresponding to a backward tilt of the patient head of 50°. At this point the centre of gravity of the first pendulum 412 lies directly above its conical pivots 422. Because the first pendulum 412 is an inverted pendulum and therefore unstable, any further motion causes it to pivot quickly away from the rest position 706 of FIG. 7a. As a result, once the minimum guide angle is achieved, the pendulum 412 and its shutter 420 pivot away from the rest position shown in FIG. 7a, to an activated position 802 in which the shutter 420 no longer lies interposed between the target 516 and the patient eye 702.

For the reasons stated previously, the second shutter 434 remains in its rest position when the device 100 is oriented between the minimum guide angle and the maximum guide angle.

When moved to the activated position, the target 516, previously invisible to the patient, is now visible. The appearance of the target 516 indicates to the patient that they have reached the minimum predetermined value of head tilt suitable for dosing, in the present example, 50°.

The highly visible target 516 also draws the patient's eye upwards in its orbit, opening the eye as described previously, for improved accessibility to the eye by a droplet of medication. Hence the selectively visible indicator 516 provides at least two roles, informing the patient that the head and device are correctly inclined for dosing, and correctly orienting the eye 702 within its orbit for dosing.

The patient now depresses the actuation lever 114 to deliver a droplet of medication to the eye, as set forth previously.

As shown at FIG. 8b, movement of the first pendulum towards the activated position moves the aperture 426 formed in the first pendulum 412 into register with the housing window 408 (not shown) and with the indentation 436 formed in the second pendulum 414. As a consequence, the tick symbol 438 located within the indentation is made externally visible via the housing aperture 408, and also via an aligned aperture 804 formed in the light-guide panel 202. This indicates to the third party that the patient head is correctly oriented beyond the minimum predetermined backward angle of tilt suitable for delivery of a drug to the patient eye 702. Hence the third party can inform the patient to then deliver the drug, as for example when teaching a patient to use the device, or the third party can themselves commence delivery of the drug by depressing the actuation lever 114 if the patient is unable to do so.

FIGS. 9a and 9b show the device 100 and indicator assembly 410 in a third orientation, when the patient head is tilted backwards beyond an angle of 60° to the vertical. Again, the patient has placed the eyecup around the eye 702 to align the dropper device 100 relative to the patient head 704 in the single predetermined position thereto.

As the patient head moves past the 60° angle, the device 100 is tilted to a maximum guide angle at which the centre of gravity of the second pendulum 414 lies directly above its conical pivots 430. Because the second pendulum 414 is an inverted pendulum, any subsequent movement of the device 100 beyond this angle causes the pendulum 414 to swing abruptly forward under gravity, pivoting so that the second shutter 434 moves to an activated position 902 in which it obscures the line of sight between the target 516 and the patient eye 702. This results in the target 516 being rendered invisible to the patient which indicates to the patient that their head is now tilted back too far, i.e. beyond the maximum predetermined backward tilt for correctly receiving a droplet of medication from the device 100. The patient is therefore able to determine that they should not actuate the device 100 until the head is tilted forward to a point where the target 516 is once again visible.

In the third orientation shown in FIGS. 9a and 9b the tick symbol 438, mounted on the second pendulum 414, moves out of register with the first pendulum aperture 426 and housing aperture 408 and is therefore no longer externally visible to a third party. Hence the third party is able to determine that the patient head is tilted at too great an angle for optimal delivery of the drug. The third party can then inform the patient to lower their head, for example when teaching a patient to use the device, or will know to withhold from operating the MLDD 310 via the actuator lever 114 in the event that they are also operating the device 100 for the patient.

In the present example, the preferred range of tilt of the patients head is from 50° to 60°. It will be understood, however, that the device 100 can be adapted so that the range of angles over which the target is visible may be different to that described above. In a further preferred embodiment, the preferred range is 10°, centred about a mid-value of 45°, for optimal patient comfort, i.e. 40° to 50°.

For the avoidance of doubt, it will be understood that moving the device from the “over-tilt” position of FIGS. 9a and 9b to the optimal range exemplified in FIGS. 8a and 8b will cause the second shutter 414 to return to its rest state 708. This will have the consequence that the target 516 is once again visible to the patient, and the tick symbol 438 is visible via the housing aperture 408 to a third party. Similarly movement of the device from the optimal orientation shown in FIGS. 8a and 8b to the “under-tilt” position of FIGS. 7a and 7b will cause the first shutter 412 to return, under gravity, to its rest position 706. This has the consequence that the first shutter 412 obscures the target 516, and the tick symbol 438 is no longer visible via the housing aperture 408 to a third party. In other words the device 100 reliably indicates that actuation should be made only in the range of predetermined values of tilt, whichever way the device 100 is being rotated i.e. towards the horizontal orientation, or towards the vertical rest orientation. The conical pivots 422, 430 and recesses 506, 510 minimise hysteresis during operation of the pendulums, ensuring that switching between the rest state 706, 708 and activated states 802, 902 occurs reliably when moving in and out of the dispensing range defined between the maximum and minimum guide angle of the device.

The device 100 is optionally provided with a mechanical interlink between the indicator assembly 410 and the actuation mechanism 338, 114 which prevents actuation of the MLDD 310 when the device 100 does not lie between the maximum and minimum guide angles. It is still beneficial to provide an indicator 516 which is visible to the patient and/or a third party to prevent the interlock from being damaged by attempted use outside of the maximum and minimum guide angles.

It will be further understood that, in the present embodiment, the two pendulums 412, 414 provide a moveable shroud 412, 414 which renders the indicator 516 selectively visible to indicate when the patient head is tilted between a predetermined range of angles suitable for delivery.

However, the moveable shroud 412, 414 could be simplified by the omission of the second pendulum 414 so that the device 100 only indicates when the threshold value i.e. lower range of the predetermined range of angles has been achieved. This simplifies construction albeit at the risk of the patient over-tilting their head during drug delivery, however, this poses less of a misuse risk than failing to reach a minimum required head tilt.

It will also be understood that, although the present device 100 provides selectively visible indication to both the patient and a third party e.g. caregiver, the device can be simplified so that it provides indication to only the patient, or only the third party. However, an advantage of the present embodiment is that the device can be used as a training aid, as a third party is able to assess whether or not the patient is correctly using the device to self-administer medication using the device by observing the window formed in the device housing.

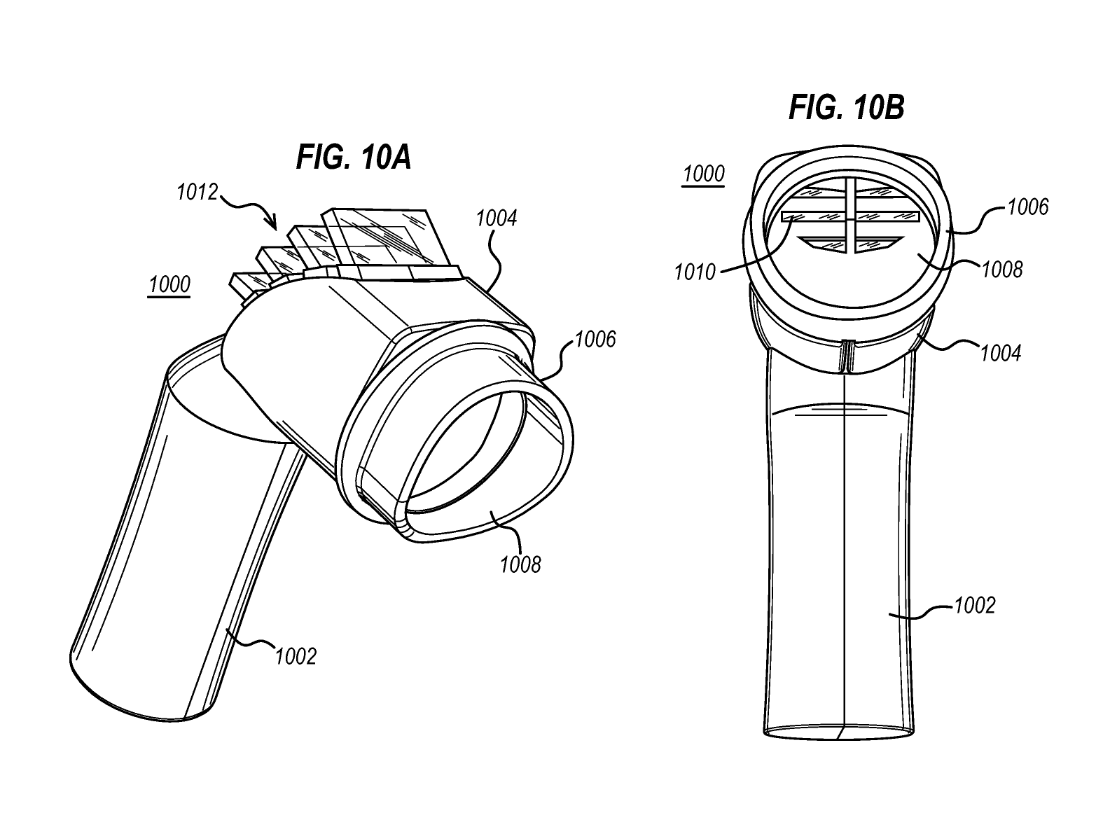

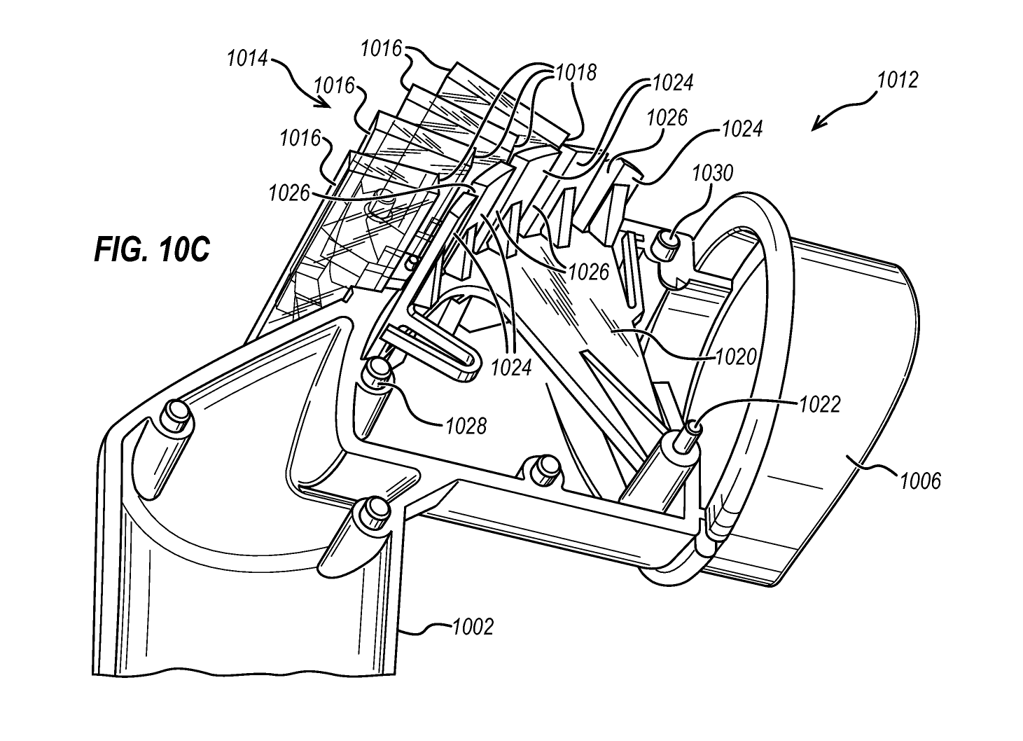

Referring now to FIGS. 10a, 10b and 10c, there is shown a second device comprising an alternative indicator assembly suitable for use in a guide device according to the present invention.

FIG. 10A shows a perspective view on a prototype device 1000 comprising a handle 1002 and an indicator assembly housing 1004. The indicator assembly further comprises an eyecup 1006 which is open to a cavity 1008, defined by the indicator assembly housing 1004.

With reference to FIG. 10B, there is shown a second perspective view on the device, which shows a view on the cavity such that a selectively visible indicator 1010, located within the cavity, is visible.

Referring now to FIG. 10C, there is shown a perspective view on the upper part of the device, providing the indicator assembly 1012. The device is sectioned so that the indicator assembly is visible.

The indicator assembly 1012 comprises an array 1014 of four planar light-guides 1016 which are inclined to converge approximately at the focus of the patient's eye (not shown), when the eye is presented to the eyecup 1006. Each light-guide 1016 comprises a rectilinear slab, which presents a single planar face 1018 to the patient eye. Each planar face 1018 is illuminated by ambient light caught by the external surface of the rest of the light guide to form an indicator surface 1018.

The indicator assembly 1012 further comprises a shutter 1020 which is mounted for pivoting motion about an axle 1022 located in the lower part of the cavity, towards the eyecup 1006.

At a distal point from the axle 1022, the shutter 1020 is provided with a vertical array of five horizontally extending slats 1024. These are spaced apart to define four horizontally elongate apertures 1026 which can be aligned with the indicator surfaces 1018 of the planar light guides 1016.

The shutter 1020 is arranged as an inverse pendulum, which is to say that the centre of gravity of the shutter 1020 lies above the shutter pivot 1022 when the device 1000 is in use.

The centre of gravity of the shutter 1020 is located such that, when the device 1000 is placed against the patient head with the head in the upright position, gravity pulls the shutter 1020 against a first stop 1028 located within the cavity towards the handle 1002. In this first “rest” position, each of the shutter slats 1024 lies interposed between a light guide 1018 and the patient eye so that the indicators 1018 are invisible to the patient.

As the patient head is tilted backwards to a first predetermined angle, the device 1000 is also tilted out of the vertical so that the centre of gravity of the shutter 1020 moves to a point above the axle 1022 of the shutter. At this point, because the shutter 1020 is an inverted pendulum and therefore unstable, any further movement causes the shutter 1020 to fall away from the rest position, pivoting about the axle 1022 to rest against a second stop 1030, provided within the cavity. In this second, activated, position, the apertures 1026 of the shutter 1020, are aligned with the indicator faces 1018 of the light-guides 1016, which are thus made visible to the user. This indicates to the user that they have attained the minimum predetermined angle of head tilt.

With reference to FIG. 10B, it can be seen that the indicators 1018 provide a large illuminated surface located towards the upper part of the eye cavity 1008. This directs the patient's eye upwards within the cavity 1008, causing the patient to maximise the exposed area of the eye and also raising the eyelashes of the upper eyelid upwards.

As shown, the device 1000 does not provide a mount suitable for holding a dispenser, or an orifice suitable for an outlet of such a dispenser. Nevertheless, the device provides a selectively visible indicator, which is suitable for the device of FIGS. 1 through 9b, in conjunction with a suitable mount for holding a dispenser relative to the indicator assembly 1012.

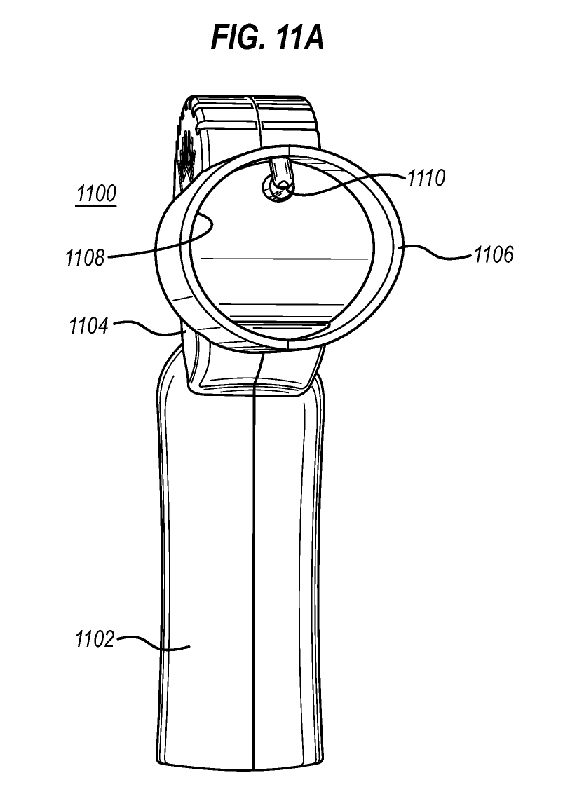

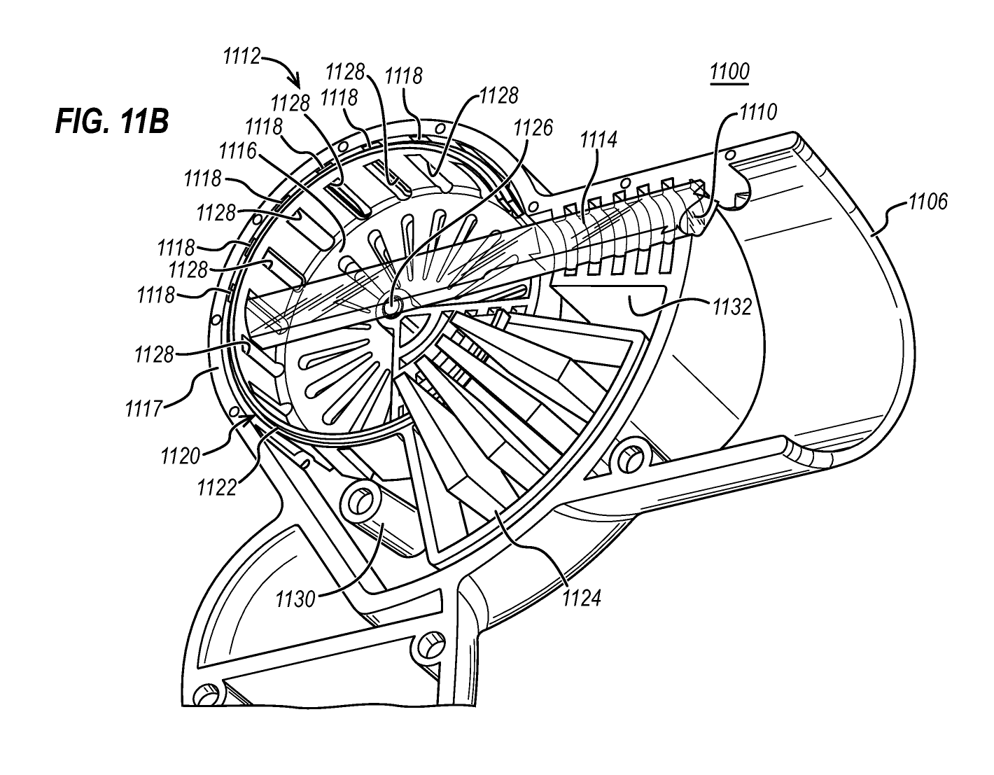

FIG. 11A shows a perspective view on a second prototype device 1100 comprising a handle 1102 and an indicator assembly housing 1104. The indicator assembly housing further comprises an eyecup 1106 which defines a cavity 1108. A selectively visible indicator 1110, located within the cavity, is visible.

Referring now to FIG. 11B, there is shown a perspective view on the upper end of the device 1100, sectioned so that an indicator assembly 1112, housed within the housing 1104, is visible.

The indicator assembly 1112 comprises a single light-guide pipe 1114 which extends across a pendulum receiving cavity 1116, defined by the indicator assembly housing 1104. A distal end of the light-guide 1114 is provided with a planar end 1110 which projects into the eye piece cavity 1108 to define the indicator 1110.

The pendulum receiving cavity 1116 is partially defined by an arcuate wall 117 in which are formed a series of equi-spaced external elongate apertures 1118.

The indicator assembly 1112 further comprises a pendulum 1120 comprising a hollow shutter drum 1122 and a weight 1124. The shutter drum 1122 is pivoted within the drum shaped housing 1104, about a central axle 1126 lies coaxial with the arcuate wall 1117 of the pendulum receiving cavity 1116. The axle 1126 allows pivoting motion of the pendulum 1120 about the shutter drum's axis of symmetry. The shutter drum 1122 is interposed between the arcuate wall 1117 and the light-guide 1114, with the weight 1124 located beneath the shutter drum 1122 so that the coaxial pendulum 1120 forms a conventional pendulum 1120.

The shutter drum 1122 has an annular wall provided with a number of elongate apertures 1128. These are of similar size and spacing to the external apertures 1118 formed in the arcuate wall 1127 of the pendulum receiving cavity 1116.

The weight 1124 of the pendulum 1120 is located beneath the shutter drum axle 1126 such that, when the device 1100 is placed against the patient head with the head in the upright position, gravity pulls the shutter 1122 into a first rest position wherein the weight 1124 rests against a first stop 1130. The stop is located within the cavity 1116 towards the handle 1102. In this first “rest” position, the apertures 1128 of the drum shutter 1122 lie out of register with the apertures 1118 formed in the housing 1104 so that light cannot enter the indicator assembly housing 1104.

As a result, the indicator 1110 is not visible when the patient eye is pressed against the eyecup 1106, as there is no ambient light passing through the light guide 1114 to the planar face 1110 of the indicator.

As the patient head is tilted backwards to a predetermined angle, the device 1100 is also tilted backwards due to the fixed relationship between the device 1100 and the patient head arising from the cooperation of the eyecup 1106 against the patient head. As a consequence, the pendulum weight 1124 causes the shutter drum 1122 to rotate to a second position in which the shutter apertures 1128 lie in register with the housing apertures 1118, as shown in FIG. 11B. This allows ambient light to enter the indicator assembly cavity 1116 and fall onto the light-guide 1114, which then channels it to the indicator face 1110, which is illuminated by the scattering of the gathered light.

Hence when the device 1100 is inclined at the predetermined angle, the indicator 1110 is illuminated, rendering the indicator 1110 visible in order to indicate to the patient they have inclined their head at the correct predetermined angle.

In the event that the patient tilts their head beyond the predetermined angle, so that the device 1100 is tilted beyond the angle to the vertical shown in FIG. 11b, the weight 1124 rotates the shutter 1122 to a third position in which the weight 1124 bears against a second stop 1132 formed by an internal surface of the device 1100.

In this third position of the shutter 1122, the housing apertures 1118 and shutter apertures 1128 lie out of register so that ambient light cannot reach the light-guide 1114. This causes the indicator 1110 to once again become invisible to the patient, indicating to them that they have exceeded the predetermined backward tilt of their head.

With reference to FIG. 11A, it can be seen that the indicator 1110 provides an illuminated surface located towards the upper part of the eye cavity 1108 when the patient head is correctly tilted, i.e. when the device 1100 is tilted as shown in FIG. 11B. This directs the patient's eye upwards within the cavity 1108, causing the patient to maximise the exposed area of the eye and also raising the eyelashes of the upper eyelid upwards.

As shown, the device 1100 does not provide a mount suitable for holding a dispenser, or an orifice suitable for an outlet of such a dispenser. Nevertheless, the device 1100 provides a selectively visible indicator 1110, which is suitable for the device of FIGS. 1 through 9b, in conjunction with a suitable mount for holding a dispenser relative to the indicator assembly 1112.

What is claimed is:

1. A guide device for assisting the application of a liquid from a liquid dispenser to an eye of a subject comprising;
a mount, to hold the liquid dispenser in the guide device,
a rest, to align the guide device with the subject head such that the mount locates an outlet of the dispenser in fixed relationship with the subject head,
an indicator, which is selectively visible to indicate when the guide device is tilted at or beyond a minimum guide angle,
wherein said minimum guide angle corresponds, when the guide device is aligned with the subject head via the rest, with a minimum predetermined tilt of the subject head suitable for delivery of the liquid from the dispenser outlet to the subject eye.

2. A guide device as claimed in claim 1 wherein the indicator is selectively visible to indicate when the guide device is tilted at or between the minimum guide angle and a maximum guide angle, wherein the maximum guide angle corresponds, when the guide device is aligned with the subject head via the rest, with a maximum predetermined tilt of the subject head suitable for delivery of the liquid from the dispenser outlet to the subject eye.

3. A guide device as claimed in claim 2 wherein the selectively visible indicator is only visible when the guide device is tilted at an angle at or between the minimum guide angle and the maximum guide angle.

4. A guide device as claimed in claim 1 wherein the guide device further comprises a moveable shroud which renders the indicator selectively visible.

5. A guide device as claimed in claim 4 wherein the shroud is mounted for movement within the guide device and said movement is powered by the weight of the shroud.

6. A guide device as claimed in claim 4 wherein the shroud comprises at least one pendulum.

7. A guide device as claimed in claim 6 wherein the at least one pendulum is an inverted pendulum.

8. A guide device as claimed in claim 4, wherein the shroud further comprises a shutter.

9. A guide device as claimed in claim 4, wherein the indicator is selectively visible to the eye of the subject and wherein the shroud is selectively interposed between the subject eye and the indicator.

10. A guide device as claimed in claim 4 wherein the indicator is selectively visible to the eye of the subject.

11. A guide device as claimed in claim 1 wherein the indicator provides a target which directs the subject's gaze upwards.

12. A guide device as claimed in claim 11 wherein the indicator provides a crescent shaped target, located within the device to lie above and concentric with the outlet of a dispenser mounted within the guide device.

13. A guide device as claimed in claim 1 wherein the indicator is selectively visible to a third party.

14. A guide device as claimed in claim 1 further comprising an actuator for operating the liquid dispenser.

15. A guide device as claimed in claim 14 wherein the actuator is adapted to convert an arcuate motion applied by an actuator lever into a substantially linear motion applied to the dispenser.

16. An eye dropper device comprising the guide device of claim 1 and a liquid dispenser.

17. An eye dropper device as claimed in claim 16 wherein the dispenser is a metered droplet dispenser for dispensing metered droplets of a liquid medication.
